(19) United States
(12) Patent Application Publication
(10) Pub. No.: US 2014/0371644 A1
(43) Pub. Date: Dec. 18, 2014

(54) ORTHOSIS AND METHOD FOR THE PLACEMENT OF AN ORTHOSIS

(71) Applicant: OPED AG, Cham (CH)

(72) Inventors: Florian Erbe, Irschenberg (DE); Gero Hopmann, Riemerling (DE)

(73) Assignee: OPED AG, Cham (CH)

(21) Appl. No.: 14/370,113

(22) PCT Filed: Nov. 22, 2012
(86) PCT No.: PCT/EP2012/004830

§ 371 Date: Jul. 1, 2014

(30) Foreign Application Priority Data
Jan. 6, 2012 (DE) 10 2012 200 165.4

(51) Int. Cl. A61F 5/37

(52) U.S. Cl. CPC A61F 5/3738
USPC 602/4

(57) ABSTRACT

An orthosis (10) for supporting an arm of a person and a method for the placement of an orthosis are provided. The orthosis includes a securing device (11) for securing the orthosis to a torso of the person, and a positioning device (12) for supporting and fixing the forearm, elbow and/or upper arm, relative to a person's shoulder associated to that arm. The positioning device is supported by the securing device. The securing device includes an abdominal strap device (13) and a shoulder strap device (14). The abdominal strap device surrounds the torso in a transversal plane, and the shoulder strap device runs over a person's shoulder opposite of that arm. The shoulder strap device includes a shoulder strap (23) and a connected chest strap (22). The straps are hinged to ends (24, 29) of the positioning device and to a ventral portion (26) of the abdominal strap device.

### CROSS REFERENCE TO RELATED APPLICATIONS

This application is a United States National Phase Application of International Application PCT/EP2012/004830 filed Nov. 22, 2012 and claims the benefit of priority under 35 U.S.C. §119 of German Patent Application DE 10 2012 200 165.4 filed Jan. 6, 2012, the entire contents of which are incorporated herein by reference.

### FIELD OF THE INVENTION

The invention relates to an orthosis for supporting an arm of a person and to a method for the placement of an orthosis, the orthosis comprising a securing device for securing the orthosis to a torso of a person, and a positioning device for supporting and fixing the arm, in particular the forearm, elbow and/or upper arm, relative to a person's shoulder associated to that arm, the positioning device being supported by the securing device, the securing device comprising an abdominal strap device and a shoulder strap device, the abdominal strap device surrounding the torso in a transversal plane when the orthosis is placed on a person, and the shoulder strap device running over the person's shoulder opposite of that arm.

### BACKGROUND OF THE INVENTION

Orthoses of this kind serve to stabilize, relieve, immobilize, guide or correct a shoulder or rather a shoulder joint of a person. To that effect, a positioning device of the orthosis is always formed in a manner that fixes a person's arm associated with the respective shoulder relative to the shoulder or that restrains the mobility of the arm so as to stabilize the shoulder or to treat a malposition of the shoulder, for example. An arm is usually fixed by means of an arm rest that runs at least in portions along a forearm. Furthermore, the known orthoses comprise a securing device that serves to connect the positioning device to a torso of the respective person. The securing device is usually formed by belts or straps that can be adjusted to the body proportions of the respective person and which secure the positioning device, i.e. the arm rest with the person's arm fixed thereto, to the torso and/or relative to the torso in the desired position.

Since oftentimes a person to be treated with an orthosis of this kind will have to wear the orthosis over a comparatively long time of several weeks, it is necessary for the orthosis to be easy to handle by the person himself/herself and for the person to be restricted as little as possible in his/her way of living. In particular, the straps or belts running on the body of the person are often considered bothersome. For example, if the orthosis also has to be worn in bed at night, the belts or straps may substantially restrict the freedom of movement and cause uncomfortable pressure marks on the person's body. The orthosis may also be formed in such a way that, for example, for a change of clothes, the respective person cannot take the orthosis off and put it back on without the help of another person. This is caused in particular by the fact that the arm associated with the treated shoulder cannot be used for this action and thus a placement of the orthosis can only be performed with one hand. The same applies to potential adjustment devices of the orthosis for the purpose of changing the position of the arm to be fixed.

### SUMMARY OF THE INVENTION

Therefore, it is an object of the present invention to provide an orthosis for supporting an arm of a person and a method for the placement of such an orthosis that allow a person to use the orthosis in a more comfortable manner.

The invention relates to an orthosis for supporting an arm of a person, comprising a securing device for securing the orthosis to a torso of the person, and a positioning device for supporting and fixing the arm, in particular the forearm, elbow and/or upper arm, relative to a person's shoulder associated to that arm, the positioning device being supported by the securing device, the securing device comprising an abdominal strap device and a shoulder strap device, the abdominal strap device surrounding the torso in a transversal plane when the orthosis is placed on a person, the shoulder strap device running over a person's shoulder opposite of that arm, the shoulder strap device comprising a shoulder strap and a chest strap connected to said shoulder strap, said straps being hinged to a distal end and to a proximal end of the positioning device and to a ventral portion of the abdominal strap device.

In consequence, the securing device comprises an abdominal strap device, a shoulder strap and a chest strap. The abdominal strap device is formed such that the orthosis placed on a person substantially surrounds the torso of a person in the area of the abdomen or of the hips. Moreover, the abdominal strap device is connected to the positioning device in such a way that the latter is arranged on the torso. The shoulder strap is hinged to a proximal end of the positioning device and runs transversally across the back and over the person's shoulder opposite of the fixed arm at least onto a front side of the torso. The shoulder strap device is continued in the area of the chest of the person by two strap portions that can be formed by the shoulder strap and the chest strap or solely by the chest strap. One strap portion is hinged to a distal end of the positioning device and the other strap portion is hinged to a ventral portion of the abdominal strap device. This arrangement of the respective straps gives way to the particular advantage of the shoulder strap running in the area of the back without a knot connection or buckle. Also, no other strap or belt is required below the arm lying opposite of the respective shoulder. Furthermore, the connection of the shoulder strap device to the distal end of the positioning device allows tensioning the shoulder strap device and thus pulling the positioning device or the arm rest closer to the torso. The connection of the shoulder strap device to the ventral portion of the abdominal strap device advantageously influences a path of the shoulder strap and of the chest strap in the area of the chest. This is particularly important if the orthosis is to be worn by female persons because they often perceive straps running across the chest to be bothersome. Moreover, it is possible owing to the afore-described design of the shoulder strap device to place or take off the orthosis one-handedly in a particularly easy manner without needing another person.

In one embodiment, the chest strap can run between the distal end of the positioning device and the ventral portion of the abdominal strap device. In this way, the shoulder strap can be hinged to the chest strap in the area of a person's chest. For example, a connecting portion of the shoulder strap and of the chest strap can then be placed in the chest area by means of corresponding length variations of the strap portions in such a manner that the shoulder strap device is not bothersome.

Further, the shoulder strap can run between the proximal end of the positioning device and the chest strap. Thus, it becomes possible to influence a strap length of the shoulder strap device between the distal end and the proximal end of the positioning device by adjusting the length of the shoulder strap or of the chest strap.

It is particularly advantageous if the shoulder strap is arranged on the connected chest strap so as to be displaceable along the chest strap. The orthosis can then be placed particularly easily in a one-handed manner if a person first places the shoulder strap over the shoulder opposite of the fixed arm. This can be done by slipping into the shoulder strap like into a jacket. Since the shoulder strap can then be arranged in a displaceable manner on the chest strap, the shoulder strap device is not yet tensioned, which makes placing the shoulder strap easier. Once the shoulder strap has been placed, the chest strap can be attached to the distal end of the positioning device. Due to this attachment, which can be performed one-handedly, the shoulder strap is tensioned. Correspondingly, the chest strap can be length-adjustable.

Moreover, it is possible to advantageously adjust a path of the shoulder strap or of the chest strap in the chest area if the chest strap is arranged on an abdominal strap of the connected abdominal strap device in a manner displaceable along the abdominal strap in the ventral portion. In that case, the chest strap, or rather a position in which the chest strap is connected to the abdominal strap, can be displaced along the abdominal strap. In this way, the orthosis can be used in a particularly universal manner since the chest strap can always be arranged by displacement in a central area of the ventral portion or in a sagittal plane of the torso irrespective of a girth of the torso or of a person's abdomen and thus independently of a length of the abdominal strap.

The positioning device can comprise a support device that is attached to a lateral portion of the abdominal strap device, and the positioning device can further comprise an arm rest, wherein an arm of a person can be fixed to the arm rest, wherein the support device can be connected to the arm rest by means of a connecting device. If the support device is attached to a lateral portion of the abdominal strap device, it is substantially located directly below a bent forearm. For example, the support device can be formed plate-shaped and can substantially be attached to the abdominal strap device in a horizontal plane. The support device and the abdominal strap device can be connected by connecting elements that are integrally formed on or flange-mounted to the abdominal strap device. The arm rest of the positioning device can be configured such that an arm of a person can be fixed to the arm rest in a particularly easy manner. For example, the arm rest can be formed in the manner of a rail or as a trough-shaped element that accommodates the respective arm at least partially. In this way, the arm rest can also accommodate an elbow and thus ensure a defined positioning of the arm in the arm rest. Preferably, the arm rest can also comprise belts or straps, by means of which a forearm and/or upper arm can easily be fixed on the arm rest. Further, it is also possible for the arm rest to have a padding so as to avoid potential pressure marks. The connecting device can connect the arm rest with the support device, wherein the connecting device can be formed directly by the arm rest and the support device. However, the connecting device can also be an autonomous component or a module that is arranged between the arm rest and the support device.

Preferably, the connecting device can be formed such that the arm rest can be fixed in a releasable manner relative to the support device. Since the support device is largely set in its position relative to a torso by its fixation to the torso via the abdominal strap device, it is then possible to orient and fix the arm rest and thus the respective arm relative to the torso in the desired position. Preferably, securing means may be provided here, which allow for a repeated release and securing and which are easy to operate with one hand.

On the arm rest, a hand rest may be arranged that can be displaced relative to the arm rest in a longitudinal direction of the arm rest. In particular in a bent position of an arm, a hand associated with that arm can thus be supported by the hand rest. Due to the hand rest being displaceable, the hand rest can further be adjusted to an individual arm length. For example, the hand rest can be secured in a longitudinal guide of the arm rest, and it can be released, displaced and fixed by means of latching elements operable with one hand in the longitudinal direction of the arm rest.

The connecting device can be formed such that the arm rest is movable relative to the support device. In this manner, the advantage can be achieved that despite an attachment of the arm rest on the support device, an arm movement is possible within defined limits. In this way, specific muscle groups can be trained, for example, by a limited movement of the arm without the shoulder to be treated being harmed by the movement. Also, on the one hand, an arm can then be fixed completely and, on the other hand, it can be moved by choice for training purposes in a moving direction defined by the connecting device.

In this way, a linear guide can be formed by the connecting device and by the support device, wherein the linear guide may be arranged such that an abduction or adduction of the arm rest is possible relative to the support device and/or to a sagittal plane of the torso. In consequence, an upper arm can be bent or stretched relative to the torso, the arm rest then moving substantially linearly relative to the support device. A movement of the arm relative to a frontal plane of the torso is then impossible due to a limitation of the arm movement being caused by the linear guide.

If it is necessary to design the linear guide in an extendable fashion, a guide portion can be adaptable to the support device, by means of which guide portion the linear guide can be extended. If the support device is formed, for example, in the manner of a plate, another plate can easily be attached to the support device, said plate extending the support device and thus the linear guide. For example, the linear guide can be formed as a recess in the support device or as a slot-shaped passage hole. Further, latching protrusions can be formed on the linear guide, by means of which the connecting device can be latched in the desired positions and can thus be fixed.

Further, the connecting device can form a rotation guide, by means of which a rotation of the arm rest is made possible in a transversal plane of the torso. In consequence, it is not necessary to orient the arm rest parallel to or in the direction of a sagittal plane of the torso, but to fix it in different angular positions relative to the sagittal plane and/or to move it in predefined limits. Like with the linear guide, a training of muscle groups and an appropriate positioning and fixing of the respective arm becomes possible by way of the rotation guide.

Preferably, the rotation guide can allow for a rotation in a range of +30° to −30° relative to the support device and/or to a sagittal plane of the torso. Moreover, latching elements may be formed on the rotation guide, which enable latching and thus fixing of the arm rest in specific angular steps of, for example, 5°.

In order to ensure a secure position of the arm rest relative to the torso and a simple handling at the same time, the shoulder strap and the chest strap can each be connectable to the arm rest in a detachable manner. In consequence, the shoulder strap can then be attached to the proximal end of the arm rest and the chest strap can be attached to the distal end of the arm rest. A severable attachment can be realized simply by a hook, buckle or plug connection. In this way, the chest strap can be easily connected to the arm rest with one hand when placing the orthosis. Furthermore, a weight of the arm is thus supported via the shoulder strap device directly over the shoulder opposite of that arm.

In this regard, it may also be advantageous if the connecting device forms a tilting guide in such a manner that a tilting of the arm rest is possible relative to the support device and/or to a transversal plane of the torso. A forearm can then be tilted at an angle or bent or stretched relative to the transversal plane. The tilting guide thus allows for a movement of the forearm in predefined limits or for an improved adjustment of a desired position of the forearm.

A tilting of the arm rest can be made possible by an adjustment of the shoulder strap or of the chest strap. In this way, the position of the forearm relative to the transversal plane can be definitively set in a simple manner and can be adapted to individual body dimensions.

In the method according to the invention for the placement of an orthosis for supporting an arm of a person, the orthosis comprises a securing device for securing the orthosis to a torso of the person, and a positioning device for supporting and fixing the arm, the positioning device being supported by the securing device, and the securing device comprising an abdominal strap device and a shoulder strap device, the arm being fixed to the positioning device, the abdominal strap device being fixed to the torso while surrounding the latter in a transversal plane, a shoulder strap of the shoulder strap device, which is connected to a proximal end of the positioning device, being pulled over a person's shoulder opposite of that arm, and a chest strap of the shoulder strap device, which is connected to the shoulder strap and is hinged to a ventral portion of the abdominal strap device, being connected to a distal end of the positioning device.

In particular, due to the design of the shoulder strap and of the chest strap and their attachment to the abdominal strap device and to the positioning device, it becomes possible to place and take off the orthosis with one hand in a particularly easy manner without the arm supported by the orthosis being needed therefor. This can be achieved in particular in that the chest strap can be maximally extended and thus forms a loop as large as possible through which a person can slip with his/her upper body.

The arm can also be balanced by adjusting the length of the chest strap. This means that the chest strap can be set or adjusted in its length in such a manner that the arm or the arm rest is arranged or balanced such that a center of gravity of the arm or of a shoulder-arm system is positioned such that the arm rests safely on the arm rest without slipping. The arm rest can in particular be arranged horizontally.

Furthermore, reference is made to the description of the advantages of the orthosis according to the invention. In the following, a preferred embodiment of the invention is explained in more detail with reference to the enclosed drawing. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a perspective view of an orthosis;

FIG. 2 is a lateral view showing the orthosis from the left, i.e. a view of a sagittal plane;

FIG. 3 is a frontal view of the orthosis, i.e. a view of a frontal plane;

FIG. 4 is a rear view of the orthosis, i.e. a view of the frontal plane;

FIG. 5 is a top view of the orthosis, i.e. a view of a transversal plane;

FIG. 6 is a view from below of the orthosis, i.e. a view of the transversal plane;

FIG. 7 is a perspective view showing a support device of the orthosis; and

FIG. 8 is a top view of the support device of the orthosis.

### DESCRIPTION OF THE PREFERRED EMBODIMENTS

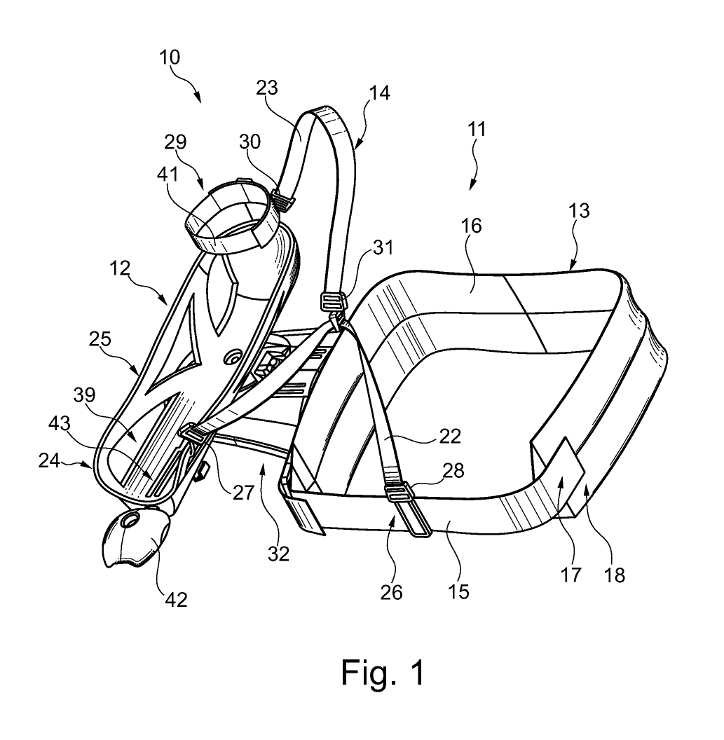

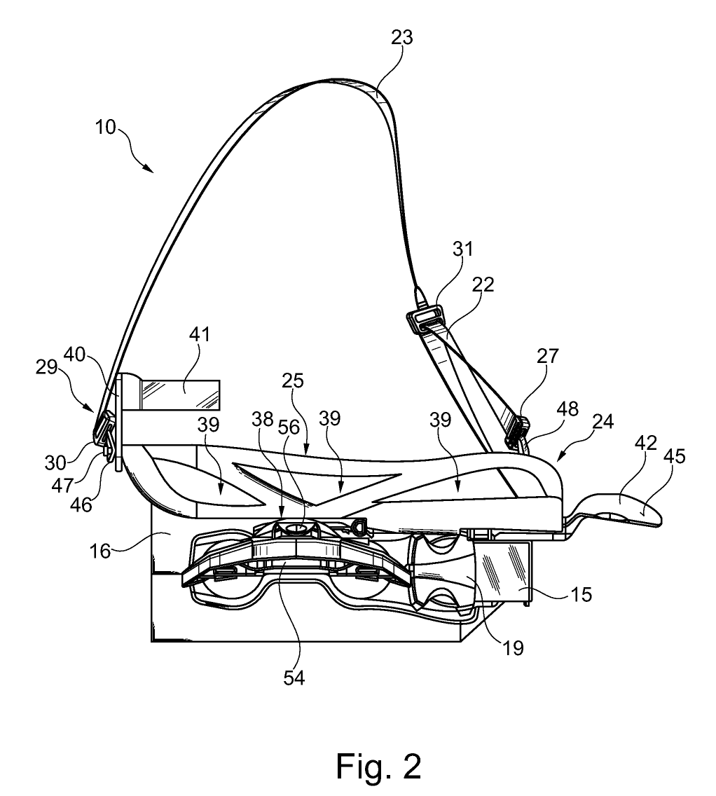

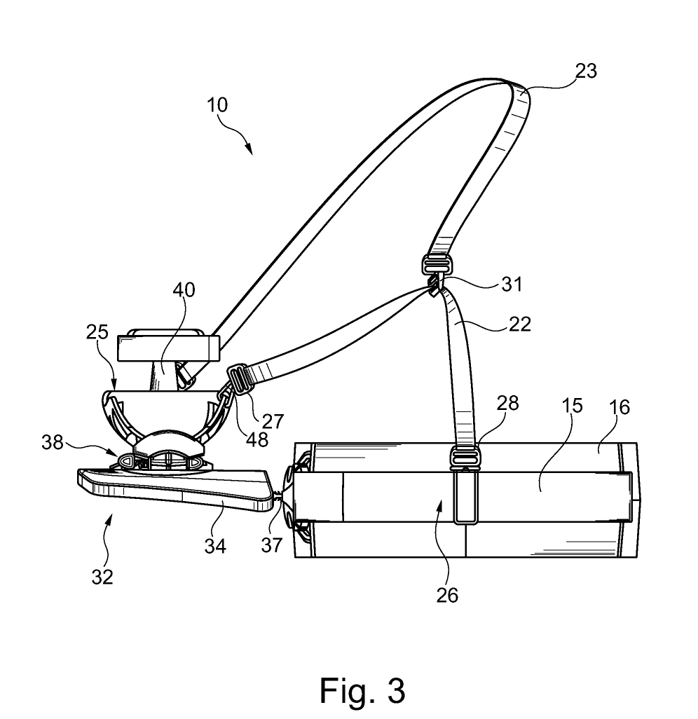

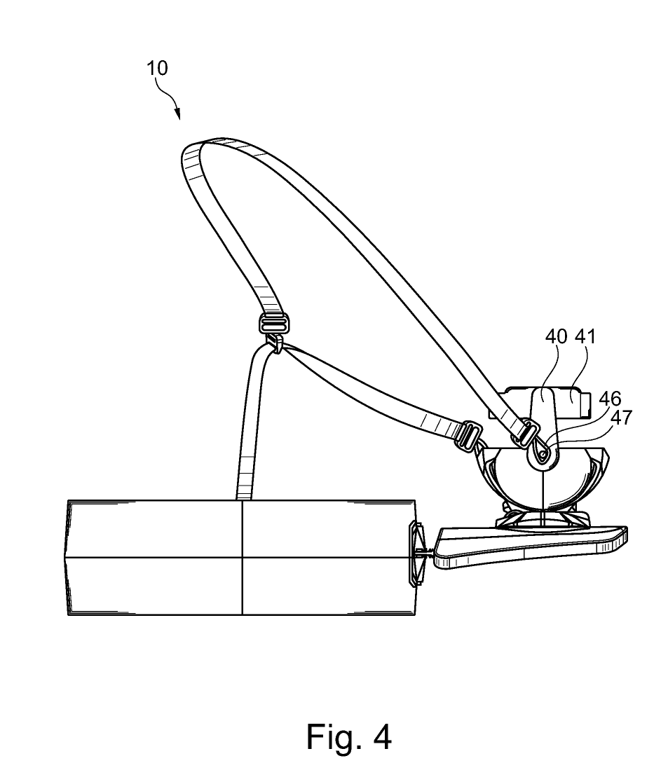

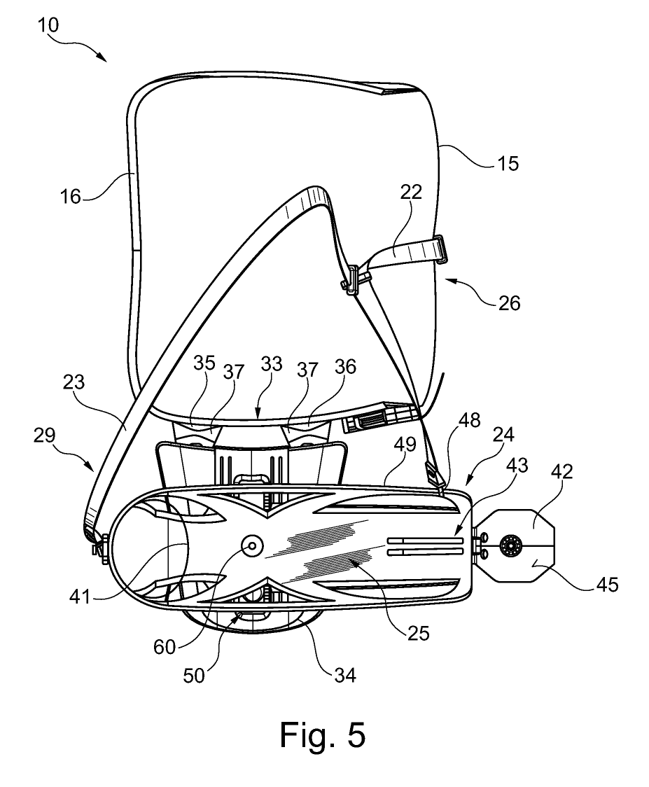

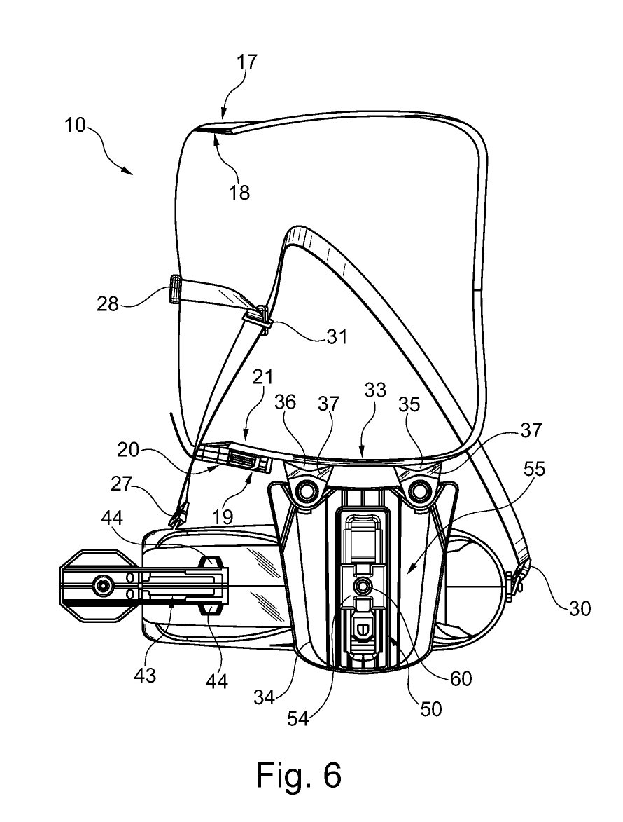

Referring to the drawings in particular, an integrated view of FIGS. 1 through 6 shows an embodiment of an orthosis 10 in different perspectives. The orthosis 10 is illustrated in the manner in which it would be arranged on a torso (not shown) of a person, i.e. placed on the person. The orthosis 10 is substantially composed of a securing device 11 and a positioning device 12, the securing device 11 serving to secure the orthosis 10 on the torso of the person, and the positioning device 12 serving to support and fix an arm (not illustrated) of the person relative to a shoulder associated with that arm. The securing device 11 supports the positioning device 12 on the torso of the person. The securing device 11 further comprises an abdominal strap device 13 and a shoulder strap device 14. The abdominal strap device 13 is formed by a flexible abdominal strap 15 that is made of a tissue material, and by a largely rigid pelvis strap 16. The pelvis strap 16 is made of a partially flexible plastic material, thus being adaptable to a body shape of the person and being able to form a comparatively rigid support for the positioning device 12 at the same time. The abdominal strap 15 is permanently fixed with one end 17 to an end 18 of the pelvis strap 16, and it is connected with a hooked buckle 19 in a releasable manner to an end 20 of the abdominal strap or to an end 21 of the pelvis strap 16. Furthermore, it is possible to vary a length of the abdominal strap 15 by means of the hooked buckle 19 and thus to adapt it to different body sizes.

The shoulder strap device 14 is substantially formed by a chest strap 22 and a shoulder strap 23. The chest strap 22 is attached to a distal end 24 of an arm rest 25 of the positioning device 12 and extends to a ventral portion 26 of the abdominal strap 15. The chest strap 22 can be attached by means of a buckle 27 to the distal end 24 of the arm rest 25, and thus it is length-adjustable. Thus, also a balancing of the arm and of the arm rest 25 is possible owing to the adjustability in length. Moreover, the chest strap 22 is connected by a buckle 28 to the abdominal strap 15, wherein the buckle 28 can be arranged in a displaceable manner along the abdominal strap 15. The buckle 28 can thus always be arranged such that it is substantially positioned in a sagittal plane of the torso. The shoulder strap 23 is attached by means of a buckle 30 to a proximal end 29 of the arm rest 25 and runs over a person's shoulder (not illustrated) opposite of that arm to the chest strap 22, and it is connected to the chest strap by a buckle 31. The shoulder strap 23 is also length-adjustable by means of the buckles 30 and 31. Further, the shoulder strap 23 can be displaced along the chest strap 22 by means of the buckle 31. Additionally, the buckles 27, 28 and 30, 31 are realized as rotating buckles, which means that they can turn about themselves so that an undesired twisting of the chest strap 22 and of the shoulder strap 23 is reversible.

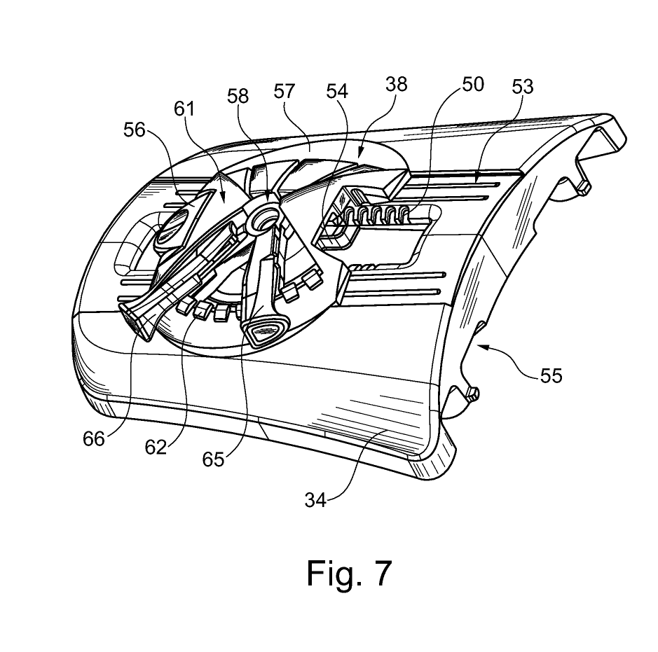

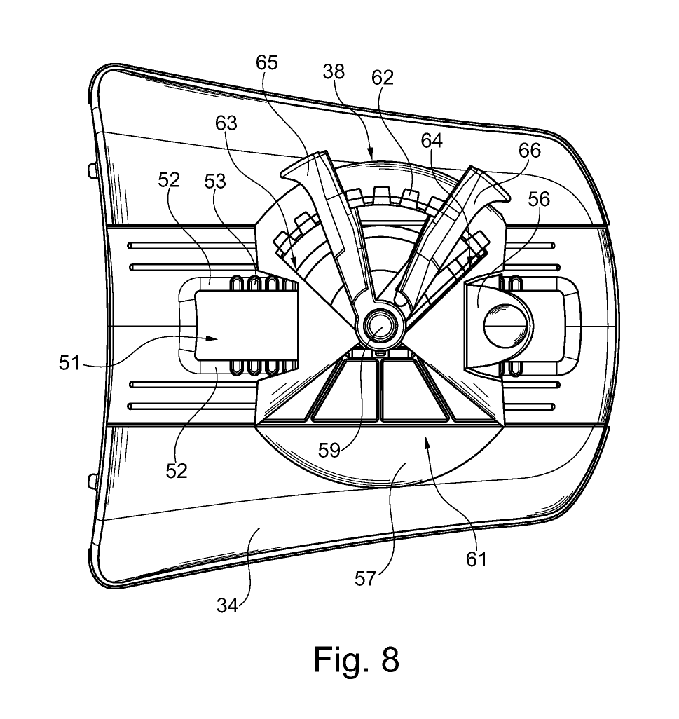

The positioning device 12 illustrated in detail in FIGS. 7 and 8 comprises a support device 32 that is attached to a lateral portion 33 of the abdominal strap device 13, i.e. to the pelvis strap 16. The support device 32 is substantially formed by a plate 34 with holding flanges 35 and 36. The holding flanges 35 and 36 are attached directly to the pelvis strap 16 and connect it to the plate 34 via one hinge 37, respectively. Thus, the plate 34 can be pivoted upwards or downwards relative to the sagittal plane of the torso. Further, the positioning device 12 comprises a connecting device 38 by means of which the arm rest 25 can be attached to the plate 34 or to the support device 32. In the example illustrated here, the arm rest 25 is formed in the shape of a trough and is thus adapted to a shape of a forearm to the largest possible extent. The proximal end 29 of the arm rest 25 is formed closed so as to accommodate an elbow in a positioning manner. Further, the arm rest 25 comprises multiple irregular through holes 39, due to which the arm rest 25 is flexible in sections. Further, it is intended that a padding (not illustrated) be inserted into the arm rest 25, which allows for a comfortable placement of the respective arm. On its proximal end 29, the arm rest 25 further comprises a shell extension 40 with an upper arm strap 41. By means of the upper arm strap 41, the upper arm and/or the elbow can be fixed in a longitudinal direction of the arm rest 25. Also, another strap (not illustrated) for fixing the forearm to the arm rest 25 may be provided at the distal end 24 of the arm rest 25. At the distal end 24 of the arm rest 25, further, a hand rest 42 is arranged that can be displaced relative to the arm rest 25 in its longitudinal direction. The hand rest 42 is accommodated in a linear guide 43 of the arm rest 25 in a manner displaceable in the longitudinal direction, wherein by means of latching noses 44 of the hand rest 42, a portioned latching and thus fixing of the hand rest 42 in the linear guide 43 is possible. For an adjustment of the hand rest 42, it thus is sufficient to compress the latching noses 44 and to simultaneously displace the hand rest 42 in the linear guide 43 into the desired position. The hand rest 42 is formed as a one-piece plastic element and has a palmar resting surface 45 for a palm.

The buckle 30 of the shoulder strap 23 is connected by a hook eye 46 to a nipple 47 formed on the proximal end 29 of the arm rest 25. The buckle 27 of the chest strap 22 is connected by way of a hook eye 48 at the distal end 24 to a wall portion 49 of the arm rest 25. When the hook eye 48 is detached, the chest strap 22 can slide through the buckle 31 in such a way that a length of the shoulder strap device 14 from the proximal end 29 to the ventral portion 26 is maximally extended, permitting the orthosis 10 to be taken off or put on in a particularly simple manner. By connecting the hook eye 48 to the wall portion 49, the shoulder strap device 14 can again be easily tensioned.

The connecting device 38 is arranged between the arm rest 25 and the plate 34. The connecting device 38 together with the plate 34 forms a linear guide 50. To that effect, a longitudinal recess 51 is formed in the plate 34, which comprises a row of latching recesses 53 on its lateral edges 52. The connecting device 38 is guided through the longitudinal recess 51 and engages behind the edges 52 by means of a clamping element 54 on an underside 55 of the plate 34. The connecting device 38 can thus be displaced in the longitudinal direction of the longitudinal recess 51 and can be fixed in the respective position by a latching element 56. The latching element 56 is hinged to a connecting device body 57 of the connecting device 38 and can latch with the latching recesses 53 by manual operation. In consequence, the latching element 56, here shown in the latched state, can be detached from the latching recesses 53 so that the connecting device body 57 can be moved freely along the longitudinal recess 51. In this way, on the one hand, the arm rest 25 can be adjusted as needed and fixed with one hand and, on the other hand, the arm rest 25 can also be moved relative to the support device 32 in the direction of the linear guide 50 for the training of specific muscle groups.

The connecting device 38 further comprises a rotating guide 58 that is substantially formed by a hub 59 in the connecting device body 57 and by an axis 60 of the arm rest 25. On a top side 61 of the connecting device body 57, starting from the hub 59, latching noses 62 are formed at intervals of 5 angular degrees. The latching noses 62 are limited by abutment flanks 63 and 64. Hence, a movement of the arm rest 25 is possible in a range of 60 angular degrees between the abutment flanks 63 and 64. In consequence, the arm rest 25 can move or rotate relative to the sagittal plane of the torso in a range of +30 and −30 angular degrees. Also, the movement of the arm rest 25 can be limited to a specific range or can be restrained completely. This is achieved by means of two abutment elements 65 and 66. The abutment elements 65 and 66 are each supported on the hub 59 so as to be rotatable, and they can be pivoted in the range of the latching noses 62 about the hub 59. Further, the abutment elements 65 and 66 can be fixed in their respective position by latching with the latching noses 62. The abutment elements 65 and 66 then limit a movement of the arm rest 25. If the abutment elements 65 and 66 rest against each other, the arm rest 25 is fixed and can no longer be rotated. The abutment elements 65 and 66 can be adjusted with one hand

While specific embodiments of the invention have been shown and described in detail to illustrate the application of the principles of the invention, it will be understood that the invention may be embodied otherwise without departing from such principles.

1. An orthosis for supporting an arm of a person, the orthosis comprising:
a securing device for securing the orthosis to a torso of the person; and
a positioning device for supporting and fixing a supported/fixed arm portion that is one of a forearm, elbow and/or upper arm, relative to a person's shoulder associated to that supported/fixed arm portion, the positioning device being supported by the securing device, the securing device comprising an abdominal strap device and a shoulder strap device, the abdominal strap device surrounding the torso in a transversal plane when the orthosis is placed on a person, and the shoulder strap device running over a person's shoulder opposite of the supported/fixed arm portion, wherein the shoulder strap device comprises straps comprising a shoulder strap and a chest strap connected to said shoulder strap, said straps being hinged to a distal end and to a proximal end of the positioning device and to a ventral portion of the abdominal strap device.

2. The orthosis according to claim 1, wherein the chest strap runs between the distal end of the positioning device and the ventral portion of the abdominal strap device.

3. The orthosis according to claim 1, wherein the shoulder strap runs between the proximal end of the positioning device and the chest strap.

4. The orthosis according to claim 1, wherein the shoulder strap is arranged on the connected chest strap so as to be displaceable along the chest strap.

5. The orthosis according to claim 1, wherein the chest strap is length-adjustable.

6. The orthosis according to claim 1, wherein the chest strap is arranged on an abdominal strap of the connected abdominal strap device so as to be displaceable along the abdominal strap in the ventral portion.

7. The orthosis according to claim 1, wherein the positioning device comprises a support device that is attached to a lateral portion of the abdominal strap device, and the positioning device comprises an arm rest, an arm of a person being fixable to the arm rest, and the support device is connected to the arm rest by means of a connecting device.

8. The orthosis according to claim 7, wherein the connecting device is formed such that the arm rest is fixed in a releasable manner relative to the support device.

9. The orthosis according to claim 7, wherein at the arm rest, a hand rest is arranged, which is displaceable relative to the arm rest in a longitudinal direction of the arm rest.

10. The orthosis according to claim 7, wherein the connecting device is formed such that the arm rest is movable relative to the support device.

11. The orthosis according to claim 7, wherein a linear guide is formed by the connecting device and the support device, said linear guide being arranged such that an abduction or adduction of the arm rest is possible relative to the support device and/or to a sagittal plane of the torso.

12. The orthosis according to claim 11, wherein a guiding portion is adaptable to the support device, by means of which guiding position the linear guide is extended.

13. The orthosis according to claim 7, wherein the connecting device forms a rotation guide in such a manner that a rotation of the arm rest is possible in a transversal plane of the torso.

14. The orthosis according to claim 13, wherein the rotation guide allows a rotation in a range from +30 degrees to −30 degrees relative to the support device and/or to a sagittal plane of the torso.

15. The orthosis according to claim 6, wherein the shoulder strap and the chest strap can each be detachably connected to the arm rest.

16. The orthosis according to claim 7, wherein the connecting device forms a tilting guide in such a manner that a tilting of the arm rest is possible relative to the support device and/or to a transversal plane of the torso.

17. The orthosis according to claim 16, wherein the tilting of the arm rest is possible by adjusting the shoulder strap or the chest strap.

18. A method for the placement of an orthosis for supporting an arm of a person, the method comprising the steps of:
providing the orthosis comprising a securing device for securing the orthosis to a torso of the person, and a positioning device for supporting and fixing the arm, the positioning device being supported by the securing device, and the securing device comprising an abdominal strap device and a shoulder strap device, wherein the arm is fixed to the positioning device, the abdominal strap device being fixed to the torso, surrounding the latter in a transversal plane;
pulling a shoulder strap of the shoulder strap device, which is connected to a proximal end of the positioning device, over the person's shoulder opposite of the arm; and
connecting a chest strap of the shoulder strap device, which is connected to the shoulder strap and is hinged to a ventral portion of the abdominal strap device to a distal end of the positioning device.

19. The method according to claim 18, wherein the arm is balanced by adjusting the length of the chest strap.
